(19) United States
(12) Patent Application Publication
(10) Pub. No.: US 2025/0356964 A1
(43) Pub. Date: Nov. 20, 2025

(54) CLINICAL TRIAL SUPPORT DEVICE, CLINICAL TRIAL SUPPORT METHOD, AND RECORDING MEDIUM

(71) Applicant: NEC Corporation, Tokyo (JP)

(72) Inventors: Remi NUMAJIRI, Tokyo (JP); Satoshi ODA, Tokyo (JP); Masahiro KUBO, Tokyo (JP)

(73) Assignee: NEC Corporation, Tokyo (JP)

(21) Appl. No.: 19/091,995

(22) Filed: Mar. 27, 2025

(30) Foreign Application Priority Data
May 17, 2024 (JP) 2024-080597

(51) Int. Cl. G16H 10/20; G16H 10/60; G16H 15/00; G16H 50/70

(52) U.S. Cl. CPC G16H 10/20; G16H 10/60; G16H 15/00; G16H 50/70

(57) ABSTRACT

A clinical trial support device includes an acquisition unit, a complementing unit, a selection unit, and an output unit. The acquisition unit acquires data regarding a treatment of a patient. The complementing unit complements missing data in the data regarding a treatment among data used to select a patient to be clinically tested. The selection unit selects a patient to be clinically tested based on the complemented data. The output unit outputs information about the selected patient to be clinically tested. With such a configuration, the clinical trial support device can support decision-making regarding selection of a patient to be clinically tested.

This application is based upon and claims the benefit of priority from Japanese Patent Application No. 2024-80597, filed on May 17, 2024 the disclosure of which is incorporated herein in its entirety by reference.

### TECHNICAL FIELD

The present disclosure relates to a clinical trial support device and the like.

### BACKGROUND ART

A system that supports selection of a candidate for a patient to be clinically tested may be used for selecting a candidate for a patient to be clinically tested. For example, a clinical trial candidate extraction device of PTL 1 (JP 2014-194595 A) stores information about a disease of a patient and information about a clinical trial drug administered to the patient. The clinical trial candidate extraction device of PTL 1 extracts a clinical trial candidate based on the number of changes of the administered clinical trial drug.

### SUMMARY

An object of the present disclosure is to provide a clinical trial support device or the like capable of efficiently selecting a patient to be clinically tested.

A clinical trial support device according to an aspect of the present disclosure includes an acquisition unit that acquires data regarding a treatment of a patient, a complementing unit that complements missing data in the data regarding the treatment among data used for selecting a patient to be clinically tested using a graph indicating a relationship between patients generated based on the data regarding the treatment of the patient, a selection unit that selects a patient to be clinically tested based on the complemented data, and an output unit that outputs information about the selected patient to be clinically tested.

A clinical trial support method according to an aspect of the present disclosure includes acquiring data regarding a treatment of a patient, complementing missing data in the data regarding the treatment among data to be used for selecting a patient to be clinically tested, selecting a patient to be clinically tested based on the complemented data, and outputting information about the selected patient to be clinically tested.

A non-transitory recording medium according to an aspect of the present disclosure records a clinical trial support program for causing a computer to execute the steps of acquiring data regarding a treatment of a patient, complementing missing data in the data regarding the treatment among data to be used for selecting a patient to be clinically tested, selecting a patient to be clinically tested based on the complemented data, and outputting information about the selected patient to be clinically tested.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of a clinical trial support system in the present disclosure;

FIG. 2 is a diagram illustrating an example of a configuration of a clinical trial support device in the present disclosure;

FIG. 3 is a diagram illustrating an example of a criterion for including a patient in a clinical trial in the present disclosure;

FIG. 4 is a diagram illustrating an example of a criterion for excluding a patient from a clinical trial in the present disclosure;

FIG. 5 is a diagram illustrating an example of a graph indicating a relationship between patients in the present disclosure;

FIG. 6 is a diagram illustrating an example of an extraction result of a patient to be clinically tested in the present disclosure;

FIG. 7 is a diagram illustrating an example of an extraction result of a patient to be clinically tested in the present disclosure;

FIG. 8 is a diagram illustrating an example of an operation flow of the clinical trial support device in the present disclosure; and

FIG. 9 is a diagram illustrating an example of a hardware configuration of the clinical trial support device in the present disclosure.

### EXAMPLE EMBODIMENT

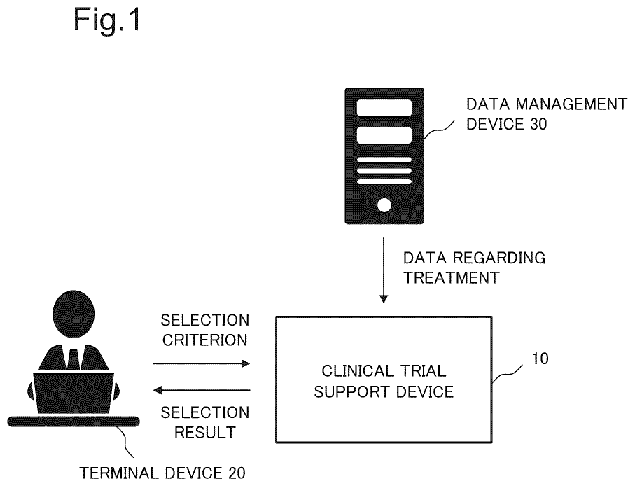

Example embodiments of the present disclosure will be described in detail with reference to the drawings. FIG. 1 is a diagram illustrating an example of a configuration of a clinical trial support system. The clinical trial support system includes a clinical trial support device 10, a terminal device 20, and a data management device 30. The clinical trial support device 10 is connected to the terminal device 20 via, for example, a network. The clinical trial support device 10 is connected to the data management device 30 via, for example, a network. A plurality of terminal devices 20 and a plurality of data management devices 30 may be provided. The number of terminal devices 20 and the number of data management devices 30 can be appropriately set.

The clinical trial support system is, for example, a system that supports selection of a patient to be clinically tested. The patient to be clinically tested is, for example, a patient to be subjected to a clinical trial of a new medicine. The clinical trial support system outputs, for example, information about a patient to be clinically tested selected from patients undergoing the treatment at a medical institution. A patient undergoing the treatment at a medical institution may include a patient who had undergone the treatment.

The patient selected as a patient to be clinically tested is, for example, a patient with a disease condition suitable for verifying the effect of the medicine. For example, the patient selected as a patient to be clinically tested is a patient whose data regarding the treatment meets the criterion for selecting a clinical test patient. The selection criterion is a condition for selecting a patient as a clinical test patient. The selection criterion includes, for example, an inclusion criterion and an exclusion criterion. The inclusion criterion indicates, for example, a criterion for including a patient in a clinical trial. The exclusion criterion indicates, for example, a criterion for excluding a patient from a clinical trial. That is, the exclusion criterion is, for example, a criterion for not selecting a patient as a clinical test patient.

In the case of selecting a patient to be clinically tested, even a patient with a similar case may not be included in a candidate when it is attempted to select a patient to be selected because part of data necessary for selecting a patient to be clinically tested is missing. For example, in a clinical trial for a medicine for an injuries and sickness with few cases, it may be difficult to select a patient suitable for the clinical trial because the number of patients that can be candidates for selection is small. For example, the clinical trial support system complements missing data among data regarding the treatment necessary for selecting a patient to be clinically tested, thereby expanding the range of patients to be selected, thereby improving the efficiency of selection of the patient to be clinically tested. For example, in an injuries and sickness with few cases, the possibility that the number of patients required for a clinical trial can be secured can be improved by complementing missing data and selecting patients to be clinically tested.

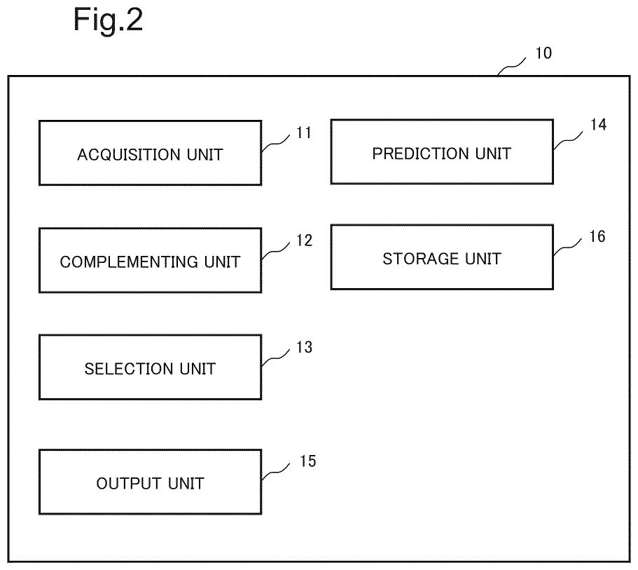

Here, an example of a configuration of the clinical trial support device 10 will be described. FIG. 2 illustrates an example of a configuration of the clinical trial support device 10. The clinical trial support device 10 includes an acquisition unit 11, a complementing unit 12, a selection unit 13, and an output unit 15 as a basic configuration. The clinical trial support device 10 may further include, for example, a prediction unit 14 and a storage unit 16.

The acquisition unit 11 acquires data regarding a treatment of a patient. For example, the acquisition unit 11 acquires data regarding a treatment of each patient who can be a subject of a clinical trial. The data regarding the treatment of the patient is, for example, the attribute of the patient and the treatment data of the patient.

The attribute of the patient is, for example, information about the state of the patient that does not change by the treatment among the information indicating the characteristics of the patient. The attribute of the patient is, for example, information of one or more items of patient gender, age, family structure, family disease history, nationality, and race. The attribute of the patient is not limited to the above.

The treatment data of the patient is, for example, a record of a medical practice performed on the patient. Patient treatment data is, for example, a record of one or more items of diagnosis, examination, medication, surgery, follow-up, and patient condition. The condition of the patient includes, for example, information of one or more items of disease information, complication information, biomarkers, disease status, guideline scores, therapeutic effects, and test results. The guideline score is, for example, an index indicating a risk in each injuries and sickness. For example, the risk in each injuries and sickness is an index indicating the possibility that the patient suffers from the injuries and sickness or the possibility that the patient is affected by the injuries and sickness. For example, the guideline score is calculated as an index indicating how much the data regarding the treatment of the patient applies to the criterion defined for each injuries and sickness. The therapeutic effect is, for example, an effect by the treatment, administration of a drug, and surgery. The effect is not limited to the above. The test result is, for example, a result of a biological test, a diagnostic imaging test, and a genome test. The test result is not limited to the above. The treatment data of the patient may include information about a person in charge who has performed the medical practice. The information about the person in charge who has performed the medical practice is, for example, information indicating a medical practitioner who has performed the medical practice on the patient. The information indicating the medical practitioner who has performed the medical practice on the patient is, for example, a name or an identifier of a doctor, a nurse, a pharmacist, and a physical therapist.

The patient treatment data is recorded as individual patient data in an electronic medical record, for example. The patient treatment data may be test data. The patient treatment data may also be data described in the health insurance claim form (health insurance claim form corresponds to, for example, the medical claim or the health insurance claim). The treatment data of the patient is not limited to the above. The data regarding the treatment is not limited to the above.

The acquisition unit 11 acquires, for example, data regarding the treatment of a patient as structured data. The structured data is, for example, data from which data can be extracted according to a rule. For example, in structured data, an item of data is associated with data in each item. In the structured data, for example, by designating an item of data, data associated with the item can be extracted. The item of data is information indicating what data each item of data is. For example, in a case where the data is an injuries and sickness name, the item is a name of the injuries and sickness.

The acquisition unit 11 may acquire data regarding the treatment of a patient as non-structural data. The non-structural data is, for example, data in an unstructured state. The non-structural data is, for example, data in which a medical practitioner expresses a state of a patient in text. The medical practitioner is, for example, a doctor. The medical practitioner may be, for example, a nurse, pharmacist, psychotherapist or physical therapist. The medical practitioner is not limited to the above. The non-structural data may be image data. The image data is, for example, imaging data in an X-ray examination, an endoscopic examination, a computed tomography (CT) examination, or a magnetic resonance imaging (MRI) examination. The image data is not limited to the above. The acquisition unit 11 acquires data regarding the treatment of a patient from the data management device 30, for example.

The acquisition unit 11 further acquires, for example, the criterion for selecting a patient to be clinically tested. For example, the acquisition unit 11 acquires at least one of the criterion for including the patient in the clinical trial and the exclusion criterion from the clinical trial as the selection criterion. The selection criterion includes, for example, a plurality of criteria used for selecting a patient to be clinically tested. The acquisition unit 11 acquires, for example, the criterion for selecting a patient to be clinically tested from the terminal device 20.

The inclusion criterion is, for example, information indicating a condition of the patient to be clinically tested. The condition of the patient to be clinically tested is indicated using attributes of the patient suitable as the clinical test patient and medical records in the treatment. The exclusion criterion is information indicating a condition for excluding the patient from the patient to be clinically tested. That is, the exclusion criterion is, for example, information indicating a condition of a patient not selected as a clinical test patient. Exclusion criterion is indicated using patient attributes and medical records in treatment that are not suitable as a clinical test patient.

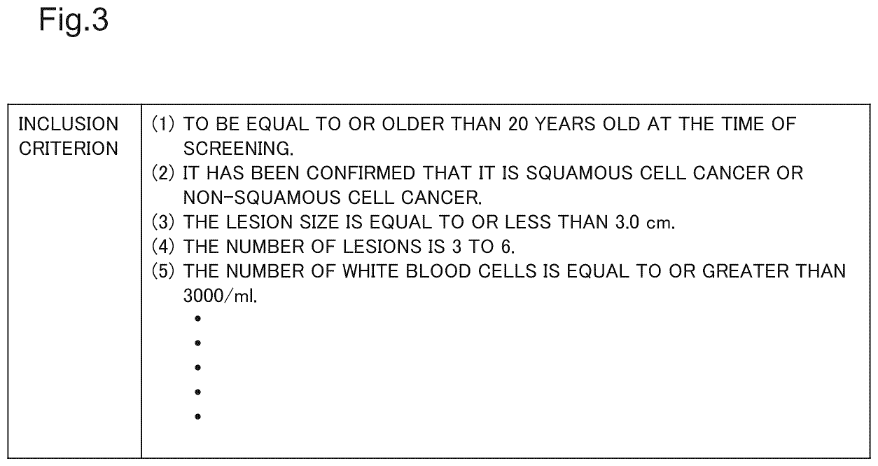

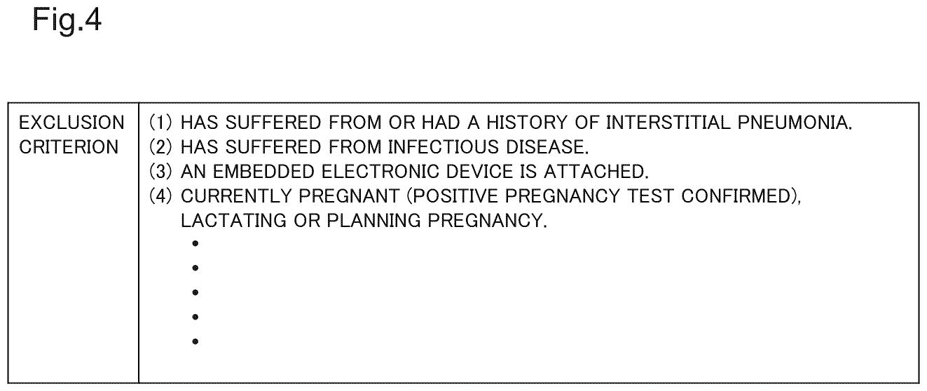

FIGS. 3 and 4 are examples of the selection criterion when conducting a clinical trial of a therapeutic agent for lung cancer. FIG. 3 is an example of a sentence indicating the criterion for including the patient in the clinical trial among the selection criterion. In the example of the inclusion criterion in the clinical trial of FIG. 3, the inclusion criterion includes a plurality of criteria. FIG. 4 is an example of a sentence indicating a criterion for excluding a patient from a clinical trial among the selection criterion. In the example of exclusion criterion from the clinical trial of FIG. 4, the exclusion criterion includes a plurality of criteria.

The acquisition unit 11 may acquire a condition prioritized in selection of a patient to be clinically tested. The prioritized condition may be a condition indicating a criterion included in the selection criterion. For example, in a case where a patient is selected with priority given to a condition related to age among the criteria included in the selection criterion, the acquisition unit 11 acquires information indicating that selection is performed with priority given to age. In a case where a patient is selected with priority given to age, the acquisition unit 11 may acquire, for example, information indicating the age to be preferentially selected among the ages indicated as the criterion in the selection criterion. The acquisition unit 11 may acquire the priority of each criterion included in the selection criterion as a condition prioritized in the selection of the patient to be clinically tested. The priority is, for example, an index indicating a degree of priority in patient selection for each criterion included in the selection criterion. The acquisition unit 11 acquires, for example, a condition prioritized in selection of a patient to be clinically tested from the terminal device 20.

The complementing unit 12 complements missing data in the data regarding the treatment among data used for selecting a patient to be clinically tested. For example, the complementing unit 12 complements missing data in the data regarding the treatment among data used for calculating goodness of fit to a criterion for selecting a patient to be clinically tested. For example, the complementing unit 12 collates the selection criterion with data regarding the treatment of each patient, and extracts a data item that is required to be complemented. For example, the complementing unit 12 complements missing data for the extracted patient for which complementation is required.

For example, the complementing unit 12 complements missing data in the data regarding the treatment using a graph indicating a relationship between patients. The graph indicating the relationship between patients includes, for example, nodes related to data regarding the respective treatments of the patients and edges related to lines connecting the patients whose data regarding the respective treatments of the patients are similar. The graph indicating the relationship between patients is generated, for example, based on data regarding the treatment of a patient. Processing of generating a graph indicating a relationship between patients will be described later.

For example, based on a graph indicating a relationship between patients, the complementing unit 12 complements missing data regarding a treatment using data of a patient whose data regarding the treatment is similar to that of a patient whose data regarding the treatment is required to be complemented. For example, the complementing unit 12 complements data of a patient in need of complementation using data regarding the treatment of a patient connected to a patient in need of complementation by an edge in a graph indicating a relationship between patients.

For example, the complementing unit 12 complements data that is required to be complemented using data of the same item among data regarding the treatment of a patient connected by an edge. For example, in a case where the data of the liver function in the blood test data among the data regarding the treatment is missing, the complementing unit 12 complements the missing data using the data of the test result of the liver function of the patient connected to the patient with the missing data with the edge.

In a case where a patient in which the data is to be complemented is connected to a plurality of patients by the edges, the complementing unit 12 may complement missing data using data of the plurality of patients connected by the edges. For example, in a case where a patient in which the data is to be complemented is connected to a plurality of patients by the edges, the complementing unit 12 complements missing data in such a way as to obtain an average of the plurality of patients connected by the edges. In a case where data is missing also in a patient connected by an edge, the complementing unit 12, for example, may complement missing data to a patient connected by an edge further using data of a patient connected by an edge. The complementing unit 12 may perform weighting based on the hierarchy of edges to complement missing data. The hierarchy of edges is, for example, the number of edges present between patients. In this case, the complementing unit 12 performs weighting in such a way that the weight of the data of the patient in the hierarchy close to the patient in which the data is to be complemented increases. In a case where a patient in which the data is to be complemented is connected to a plurality of patients by the edges, the complementing unit 12 may complement missing data using data of a patient having the highest similarity in data regarding the treatment with the patient in which the data is to be complemented among the plurality of patients connected by the edges. For example, the complementing unit 12 generates a graph indicating a relationship between patients based on data regarding the treatment of each patient. Among the data regarding the treatment, the data used for generating the graph is set according to, for example, the selection criterion. For example, the complementing unit 12 generates a graph indicating a relationship between patients by the following processing. For example, the complementing unit 12 converts the data regarding the treatment into an embedding vector using the language model for each patient. For example, the language model converts the data about the treatment into an embedding vector using a dictionary. For example, the complementing unit 12 converts the data regarding the treatment into a feature vector using the converted embedding vector. The feature vector in this case is a multi-dimensional vector reflecting data regarding the treatment of each patient. That is, the feature vector in this case is a multi-dimensional vector representing each feature of the patient.

For example, the language model converts data about the treatment of each patient into an embedding vector using a dictionary related to the general medical field. The language model may convert data about the treatment of each patient into an embedding vector using a plurality of dictionaries. For example, the language model converts data regarding the treatment of each patient into an embedding vector using a dictionary related to the general medical field and a dictionary related to a clinical department in which a drug to be clinically tested is used.

For example, Word2 Vec can be used as the language model. As the language model, for example, Generative Pre-trained Transformer-2 (GPT-2), GPT-3, GPT-3.5, or GPT-4 can be used. The text-to-text transfer transformer (T5), bidirectional encoder representations from transformers (BERT), and robustly optimized BERT approach (ROBERTa) may be used as the language model, or efficiently learning an encoder that classifies token replacements accurately (ELECTRA) may be used as the language model. The language model used for conversion into the embedding vector is not limited to the above.

For example, the complementing unit 12 calculates the similarity between the patients using the distance between the feature vectors of the respective patients. For example, the complementing unit 12 calculates the similarity between the patients by calculating the distance between the feature vectors. The distance between the vectors is, for example, a Euclidean distance. The distance between the feature vectors is not limited to the Euclidean distance. The complementing unit 12 may calculate similarity between patients by calculating cosine similarity between feature vectors. The complementing unit 12 generates a graph indicating a relationship between patients by connecting nodes with edges based on similarity between patients. For example, the complementing unit 12 generates a graph by connecting nodes of patients by the edges between patients with a similarity equal to or higher than a predetermined criterion. The predetermined criterion is set in such a way that, for example, when the similarity exceeds the predetermined criterion, the patients are considered to be similar in conducting the clinical trial. Patients being similar to each other in conducting a clinical trial means that, for example, it can be expected that the effect of a medicine for conducting a clinical trial appears similarly between patients. Here, the node indicates, for example, each patient. The edge indicates, for example, a relationship between patients. That is, the graph indicating the relationship between patients is, for example, a graph in which nodes indicating respective patients are connected by edges connecting similar patients. Being similar means, for example, that the data regarding the treatment has a relationship. That the data regarding the treatment has a relationship means that, for example, when one meets the selection criterion of the clinical trial, the other is likely to meet the selection criterion of the clinical trial. That is, being similar means that, for example, when one patient fits a certain selection criterion, the other patient is likely to fit. The similarity may include the same.

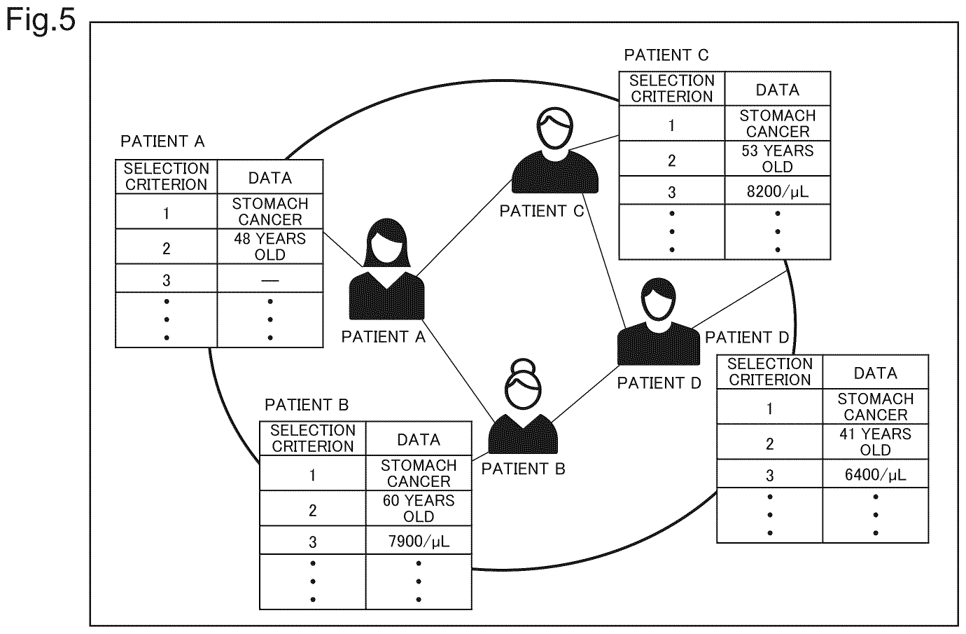

FIG. 5 is a diagram schematically illustrating an example of a graph indicating a relationship between patients generated by the complementing unit 12. In the example of FIG. 5, the graph indicating the relationship between patients includes “patient A”, “patient B”, “patient C”, and “patient D” as nodes. In the example of FIG. 5, “patient A” and “patient B”, and “patient A” and “patient C” are connected by edges. In the example of FIG. 5, “patient D” and “patient B”, and “patient D” and “patient C” are connected by edges. In the example of FIG. 5, data related to the selection criterion among the data regarding the treatment is indicated for each patient corresponding to the node. In the example of FIG. 5, “1” of the selection criterion is a criterion related to an injuries and sickness name. In the example of FIG. 5, “2” of the selection criterion is a criterion related to the age of the patient. In the example of FIG. 5, “3” of the selection criterion is test data of the number of white blood cells in blood. In the example of FIG. 5, for example, the test data of the number of white blood cells for “patient A” is missing. In a case where the data missing occurs as illustrated in the example of FIG. 5, the complementing unit 12 complements the data of the number of white blood cells of “patient A” using the test data of the number of white blood cells of each of “patient B” and “patient C” connected to “patient A” by the edges. For example, the complementing unit 12 complements the data of the number of white blood cells of “patient A” using the average value of the number of white blood cells of “patient B” and the number of white blood cells of “patient C” connected to “patient A” by the edges. The complementing unit 12 may complement the data of the number of white blood cells of “patient A” using the value of the number of white blood cells of a patient having a higher similarity with “patient A” among “patient B” and “patient C”.

The complementing unit 12 may generate a graph indicating the relationship between patients using the graph generation model. The graph generation model is, for example, a machine learning model that generates a graph indicating a relationship between patients from data regarding the treatment of each patient. For example, the complementing unit 12 converts data regarding the treatment for each patient into an embedding vector using a language model. For example, the complementing unit 12 generates a graph indicating a relationship between patients using the embedding vector as an input of the graph generation model. The graph generation model is generated, for example, by learning a relationship between data regarding the treatment of each patient and a graph indicating a relationship between patients. The graph generation model is generated by deep learning using a neural network, for example. The learning algorithm used for generating the graph generation model is not limited to the above. The graph generation model is generated, for example, in a device outside the clinical trial support device 10.

The complementing unit 12 may complement missing data using non-structural data. In a case where the non-structural data is a sentence described in the electronic medical record, for example, the complementing unit 12 complements missing data from the sentence described in the electronic medical record. In a case where the non-structural data is a sentence described in the electronic medical record, processing of complementing missing data using the non-structural data is performed as follows, for example. The complementing unit 12 identifies, for example, missing data in selecting a patient to be clinically tested. For example, the complementing unit 12 extracts a description related to the missing data from the electronic medical record using the language model based on the information indicating the missing data. As the language model, for example, Generative Pre-trained Transformer-2 (GPT-2), GPT-3, GPT-3.5, or GPT-4 can be used. The text-to-text transfer transformer (T5), bidirectional encoder representations from transformers (BERT), and robustly optimized BERT approach (ROBERTa) may be used as the language model, or efficiently learning an encoder that classifies token replacements accurately (ELECTRA) may be used as the language model. The language model used for extracting the missing data is not limited to the above. The language model used for extracting the missing data may be the language model same as the language model for converting the data regarding the treatment into the embedding vector. The language model used for extracting the missing data may be a language model different from a language model for converting data regarding the treatment into an embedding vector.

In a case where the non-structural data is image data, the complementing unit 12 complements missing data using, for example, a diagnosis result by an image diagnosis model. The image diagnosis model is, for example, a machine learning model that estimates an injuries and sickness name using image data captured in an examination as an input. The image diagnosis model may be a machine learning model that estimates the size of the lesion with the image data captured in the examination as an input. The image diagnosis model may be a machine learning model that estimates an injuries and sickness name and a size of a lesion by using image data captured in an examination as an input. For example, in a case where data of the size of a lesion in a patient with gastric cancer is missing, the complementing unit 12 estimates the size of the lesion in the stomach using the image diagnosis model based on image data in endoscopic examination of the stomach.

The image diagnosis model is generated, for example, by learning the relationship between the image data captured in the examination and the injuries and sickness name. The image diagnosis model may be generated by learning the relationship between image data captured in an examination and a range of a lesion on the image data. The image diagnosis model may be generated by learning the relationship between the image data captured in the examination and the injuries and sickness name and the range of the lesion on the image data. The image diagnosis model is generated by deep learning using a neural network, for example. The image diagnosis model is generated, for example, in a system outside the clinical trial support device 10.

In a case where the selection unit 13 selects a patient to be clinically tested based on data regarding the past treatment, the complementing unit 12 may complement missing data among the data regarding the past treatment. For example, the complementing unit 12 complements missing data for a patient having a missing data regarding the past treatment among patients for which data regarding the treatment for the implementation period of the clinical trial from the past time point is recorded. In a case where the prediction unit 14 predicts data regarding the treatment, the complementing unit 12 may complement missing data in the data regarding the treatment predicted by the prediction unit 14. For example, the prediction unit 14 interpolates missing data among prediction values of data regarding the treatment.

The complementing unit 12 may generate data of each patient that can be regarded as a state in which the missing data is complemented as data obtained by complementing the missing data. For example, the complementing unit 12 generates each feature vector of each patient as data obtained by complementing missing data in which is based on a graph indicating a relationship between patients generated based on data regarding the treatment of the patient and the criterion for selecting a patient to be clinically tested. For example, the complementing unit 12 generates the feature vector of each patient based on the graph indicating the relationship between the patients and the goodness of fit of the data regarding the treatment with respect to the selection criterion. In this case, for example, the complementing unit 12 calculates the goodness of fit of the data regarding the treatment with respect to the selection criterion. For example, the complementing unit 12 calculates the ratio of the number of criteria in which the data regarding the treatment satisfies the condition to the number of criteria included in the selection criterion as the goodness of fit of the data regarding the treatment with respect to the selection criterion.

For example, the complementing unit 12 converts a graph indicating the goodness of fit for each criterion included in the selection criterion and the relationship between patients into a feature vector of each patient. For example, the complementing unit 12 converts the graph indicating the goodness of fit with respect to the selection criterion and the relationship between the patients into feature vectors of the respective patients using a conversion model that converts the graph indicating the goodness of fit and the relationship between the patients into one feature vector. The conversion model is, for example, a machine learning model that converts a graph indicating the goodness of fit of the patient with each criterion included in the selection criterion and the relationship between the patients into a feature vector of each patient.

The conversion model is generated as follows, for example. In the first stage in the generation of the conversion model, the learning device that generates the conversion model learns the vector representation for each information about the attribute and the treatment using, for example, a message passing method of exchanging a message via an edge of a graph indicating a relationship between patients. In the second stage, the learning device learns, for example, a vector representation for each node in which vectors for information about the goodness of fit, the attribute, and the treatment are combined. The node is related to each patient, for example. The conversion model generated in this manner can convert a graph indicating the goodness of fit and the relationship between patients into a feature vector. Such a learning method is also referred to as embedding propagation. The conversion model is generated, for example, in a system outside the clinical trial support device 10. The conversion model may be generated by a learning means (not illustrated) included in the clinical trial support device 10.

The selection unit 13 selects a patient to be clinically tested based on the complemented data. For example, the selection unit 13 selects a patient to be clinically tested based on the goodness of fit of the data regarding the treatment of each patient with respect to the selection criterion. For a patient having no data missing, the selection unit 13 performs a process of selecting a patient to be clinically tested, for example, using data regarding the treatment for which processing regarding complementation has not been performed. That is, the selection unit 13 selects the patient to be clinically tested based on the data regarding the treatment of the patient in which the data has been complemented and the data regarding the treatment of the patient for which the data does not need to be complemented.

As the process of selecting the patient to be clinically tested, for example, the selection unit 13 calculates the goodness of fit of the data regarding the treatment of each patient with respect to the criterion for selecting the patient to be clinically tested. In this case, for example, the selection unit 13 calculates the goodness of fit to the selection criterion for the patient in which the data has been complemented, using the data regarding the treatment after the complementation. The goodness of fit is, for example, a ratio of the number of criteria in which the data regarding the treatment satisfies the condition to the number of criteria included in the selection criterion. The selection unit 13 may calculate the goodness of fit by weighting each criterion included in the selection criterion. The selection unit 13 selects, for example, a patient whose goodness of fit is equal to or higher than a criterion as a patient to be clinically tested. The criterion for goodness of fit when selected as the patient to be clinically tested is set in such a way that the patient in which goodness of fit exceeds the criterion is a patient as a suitable patient to be clinically tested.

The selection unit 13 may extract the goodness of fit for the clinical trial of the patient in a plurality of stages. Setting the goodness of fit in a plurality of stages makes it possible, for example, to extract a patient to be a candidate for a patient to be clinically tested when part of the criterion is relaxed. For example, the selection unit 13 extracts, for each patient, information indicating which of a plurality of stages the goodness of fit for the clinical trial estimated by the extraction model is. The selection unit 13 may extract the number of patients suitable for the clinical trial. In a case where a plurality of stages according to the goodness of fit to the clinical trial is set, the selection unit 13 may extract the number of patients for each stage.

The selection unit 13 may select the patient in the clinical trial group and the patient in the control group as the patients to be clinically tested. The patient in the clinical trial group is, for example, a patient to whom the medicine of the clinical trial is administered. The patient in the control group is, for example, a patient to whom the medicine of the clinical trial is not administered for comparison with the patient in the clinical trial group. For example, a substance called placebo is administered to the patient in the control group. The placebo is, for example, a substance that does not contain an active ingredient that is indistinguishable from the medicine of the clinical trial.

The selection unit 13 may select the patient to be clinically tested further based on the data regarding the treatment of the selected patient as the patient to be clinically tested. For example, the selection unit 13 selects a patient to be clinically tested in such a way that the selected patients to be clinically tested is not biased based on the data regarding the treatment of the selected patient to be clinically tested. The term “is not biased” means that, for example, there is no bias in the data regarding the treatment between the patients in the control group and the patients in the clinical trial group. For example, the selection unit 13 randomly selects the patients in the clinical trial group and the patients in the control group from among the patients in which goodness of fit of the data regarding the treatment with respect to the selection criterion satisfies the criterion.

The selection unit 13 may select a patient in one of the control group and the clinical trial group as the patient to be clinically tested. For example, in a case where the patient in the clinical trial group has been selected, the selection unit 13 selects the patient in the control group. In this case, for example, the selection unit 13 selects the patient in the control group in such a way that there is no bias between the patients in the clinical trial group and the patients in the control group. For example, the selection unit 13 selects a patient in the control group based on data of the selected patient in the clinical trial group as a patient to be clinically tested. For example, in a case where the selected patients of the clinical trial group are patients in their 20's and 30's, the selection unit 13 preferentially selects patients in their 20's and 30's as patients in the control group. The selection unit 13 may select some of the patients in the control group based on the data of the selected patients in the clinical trial group as the patients to be clinically tested. The selection unit 13 may select the patient in the control group based on data of the selected patient in the control group as a patient to be clinically tested.

The selection unit 13 may further select the patient in the clinical trial group based on data of the selected patient in the clinical trial group as the patient to be clinically tested. For example, the selection unit 13 further selects the patient in the clinical trial group in such a way that there is no bias in the data regarding the treatment of the patient within the criterion included in the selection criterion. For example, in a case where the selection criterion includes a condition of age from 20 to 50 years old and the selected patients in the clinical trial group are patients in their 20's and 30's, the selection unit 13 preferentially selects patients in their 40's as patients in the clinical trial group. The selection unit 13 may further select the patient in the control group based on data of the selected patient in the control group as a patient to be clinically tested.

In a case where the respective feature vectors of the patients are generated in the complementing unit 12, the selection unit 13 extracts a patient suitable for the clinical trial as a candidate for the patient to be clinically tested, for example, based on the respective feature vectors of the patients. The feature vector of each patient is a feature vector converted by the complementing unit 12 from a graph indicating the goodness of fit of each patient with respect to the selection criterion and the relationship between the patients. For example, the selection unit 13 extracts a patient whose goodness of fit to the clinical trial is equal to or higher than the criterion as a patient conforming to the clinical trial. The criterion for goodness of fit for a clinical trial is set, for example, in such a way that a patient whose goodness of fit exceeds the criterion is a patient suitable as a clinical test patient. In a case where there is a condition for the patient to be preferentially selected, the selection unit 13 may select the patient to be clinically tested by changing the related criterion among the selection criteria in such a way that the patient to be preferentially selected is selected. For example, in a case where the selection criterion includes a condition of age from 20 to 50 years old, and in a case where the selected patients are patients in their 20's and 30's, the selection unit 13 may change the criterion indicating age among the selection criteria so that the patient should be a patient in their 40's, and select a patient to be clinically tested.

The selection unit 13 extracts a candidate of a patient to be clinically tested, for example, using an extraction model. The extraction model is used, for example, to extract a patient suitable for the clinical trial based on a feature vector of each patient. The extraction model is, for example, a machine learning model that estimates a goodness of fit to a clinical trial using a feature vector of each patient as an input. The goodness of fit to the clinical trial is, for example, the probability that the patient will fit the clinical trial. The selection unit 13 extracts a patient suitable for the clinical trial, for example, based on the goodness of fit to the clinical trial of each patient estimated by the extraction model. That is, the selection unit 13 extracts, for example, a patient whose goodness of fit to each clinical trial estimated by the extraction model is equal to or higher than a criterion as a patient suitable for the clinical trial.

The extraction model is generated, for example, by learning the relationship between the feature vector of each patient and the presence or absence of conformity to the clinical trial. The feature vector of each patient is a feature vector converted from the graph indicating the goodness of fit and the relationship between patients by the complementing unit 12 using the conversion model. The presence or absence of conformity to the clinical trial is actual data of the presence or absence of conformity to the clinical trial criterion of each patient. The extraction model is generated by deep learning using a neural network, for example. The extraction model is generated in a system of a device outside the clinical trial support device 10.

The selection unit 13 may select a patient to be clinically tested based on data regarding the past treatments. The selection unit 13 may select a patient to be clinically tested based on data obtained by complementing missing data among data regarding the past treatments. For example, the selection unit 13 selects a patient to be clinically tested from among patients in which the selection criterion is met at a past time point and data regarding a treatment for an implementation period of a clinical trial from a time point at which the selection criterion is met is recorded. For example, the selection unit 13 selects the patient in the control group from among the patients to be clinically tested based on the data regarding the past treatment. Conforming to the selection criterion means that, for example, the goodness of fit of data regarding the treatment of the patient with respect to the selection criterion for selecting the patient to be clinically tested satisfies the criterion for selecting the patient to be clinically tested.

For example, the prediction unit 14 predicts data regarding the treatment of the patient at a predetermined time point based on the data regarding the treatment. For example, the prediction unit 14 predicts a future state of the patient. The predetermined time point is, for example, a time point at which the clinical trial is started. The predetermined time point may be a time point at which the patient of the clinical trial is determined. The predetermined time point may be a time point in the implementation period of the clinical trial. The predetermined time point is not limited to the above. For example, the prediction unit 14 predicts data regarding the treatment at a predetermined time point for the patient selected by the selection unit 13. The prediction result of the data regarding the treatment at a predetermined time point may be used as data to be complemented by the complementing unit 12. The prediction result of the data regarding the treatment at the predetermined time point may be used as data regarding the treatment for the selection unit 13 to extract the patient to be clinically tested.

For example, the prediction unit 14 predicts data regarding the treatment at a predetermined time point using a prediction formula for each item of data regarding the treatment. The prediction formula is, for example, a function having a prediction value of data regarding the treatment as an objective variable, data regarding the treatment at a past time point or a current time point, and an elapsed time from the past time point or the current time point as explanatory variables.

The prediction unit 14 may predict data regarding the treatment of a patient at a predetermined time point using a prediction model. The prediction model is, for example, a machine learning model that uses data regarding the treatment at a past time point or a current time point and an elapsed time from the past time point or the current time point as inputs and predicts data regarding the treatment at a predetermined time point. The prediction model is generated, for example, by learning the relationship between the data regarding the treatment at the first time point and the time from the first time point to the second time point and the data regarding the treatment at the second time point. The prediction model is generated by deep learning using a neural network, for example. The machine learning algorithm used for generating the prediction model is not limited to the above. The prediction model is generated, for example, in a device outside the clinical trial support device 10.

The output unit 15 outputs information about the selected patient to be clinically tested. The output unit 15 outputs, for example, identification information about the patient to be clinically tested selected by the selection unit 13 and data regarding the treatment of each patient. For example, the output unit 15 outputs information about the selected patient to be clinically tested. The output unit 15 outputs, for example, data regarding the treatment included in the selection criterion as data regarding the treatment of each patient.

The output unit 15 may output the goodness of fit to the clinical trial for each patient to be clinically tested selected by the selection unit 13 as the information about the selected patient to be clinically tested. The output unit 15 may output, for example, the goodness of fit of the data regarding the treatment of each patient with respect to the selection criterion as the goodness of fit to the clinical trial.

The output unit 15 may output information about the selected patient to be clinically tested for each attribute of the patient. For example, the output unit 15 outputs information about the patient to be clinically tested for each age group of the patient. For example, in a case where the subject of the clinical trial is set to be equal to or greater than 20 years old and less than 50 years old in the selection criterion, the output unit 15 outputs the number of patients to be clinically tested in each age group of 20's, 30's, and 40's.

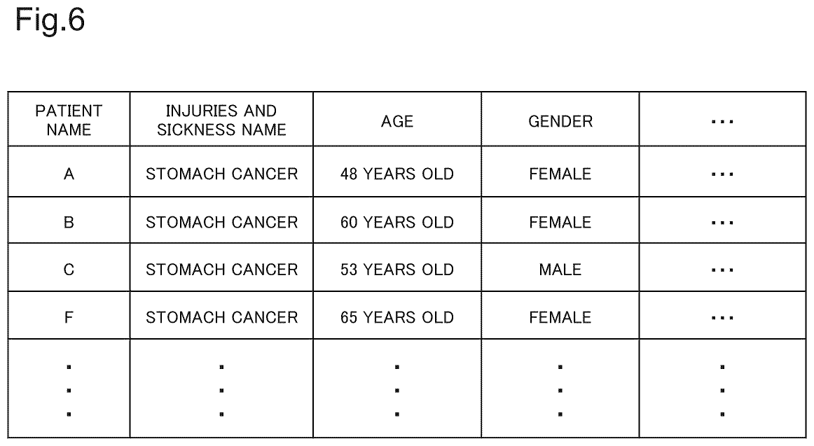

FIG. 6 is an example of a display screen displaying an extraction result of a patient to be clinically tested. In the display screen of FIG. 6, a “patient name” indicating the patient extracted as the patient to be clinically tested is associated with data regarding the treatment of each extracted patient. The “patient name” is, for example, an identifier of a patient. In the “patient name”, for example, a name, an identification number, or an identification symbol of a patient is used. In the display screen of FIG. 6, an “injuries and sickness name”, an “age”, and a “gender” among data regarding the treatment of each patient are associated with the “patient name”. The “injuries and sickness name” is, for example, an injuries and sickness name to be treated. The data regarding the treatment associated with “patient name” is not limited to the above. Since the output unit 15 outputs the extraction result of the patient to be clinically tested as illustrated in the example of the display screen of FIG. 6, the person in charge of selecting the patient to be clinically tested can determine the patient to be clinically tested while referring to the state of each patient.

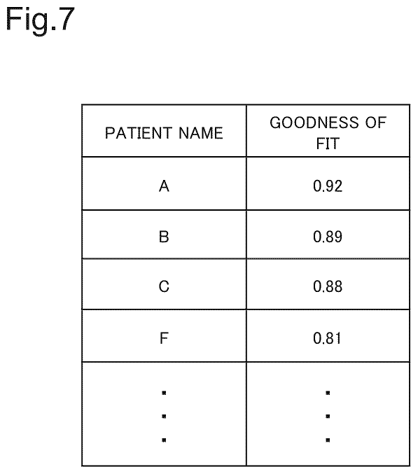

FIG. 7 is an example of a display screen that displays goodness of fit to selection criterion for each extracted patient as an extraction result of the patient to be clinically tested. In the display screen of FIG. 7, the “patient name” indicating the patient extracted as the patient to be clinically tested is associated with the goodness of fit of each extracted patient to be clinically tested. The “patient name” is, for example, an identifier of a patient. In the “patient name”, for example, a name, an identification number, or an identification symbol of a patient is used. Since the output unit 15 outputs the extraction result of the patient to be clinically tested as illustrated in the example of the display screen of FIG. 7, the person in charge of selecting the patient to be clinically tested can determine the patient to be clinically tested while referring to the goodness of fit to the selection criterion.

In a case where the prediction unit 14 predicts data regarding the treatment at a predetermined time point, the output unit 15 may output a prediction value of data regarding the treatment of the patient to be clinically tested selected by the selection unit 13. For example, the output unit 15 outputs a prediction value of data regarding the treatment at the start time point of the clinical trial for the patient to be clinically tested selected by the selection unit 13 based on the prediction result of the prediction unit 14.

The storage unit 16 stores, for example, data related to processing of selecting a patient to be clinically tested. The storage unit 16 stores, for example, data regarding the treatment of each patient acquired by the acquisition unit 11. The storage unit 16 stores, for example, information indicating a patient to be clinically tested selected by the selection unit 13. The storage unit 16 stores, for example, the criterion for selecting a patient to be clinically tested. The storage unit 16 stores, for example, the selection model. The storage unit 16 stores, for example, the graph generation model. The storage unit 16 stores, for example, the image diagnosis model. The storage unit 16 stores, for example, the prediction model. The selection model, the graph generation model, the image diagnosis model, and the prediction model may be stored in the storage means of the storage unit 16.

The terminal device 20 is, for example, a terminal device used for processing of accessing the clinical trial support device 10 and extracting a patient to be clinically tested. The terminal device 20 is, for example, a terminal device used by a person in charge of a medical institution or a person in charge of an institution commissioned by the medical institution to conduct the clinical trial. The organization to which the hospital entrusts the handling of the clinical trial is, for example, a site management organization (SMO). The person in charge of an institution that has been commissioned by a medical institution to conduct the clinical trial is, for example, a clinical research coordinator (CRC). The terminal device 20 may be a terminal device used by a person in charge who performs a clinical trial in a medicine company or a person in charge in an institution entrusted with a clinical trial by a pharmaceutical company that performs a medicine clinical trial. The institution entrusted with the clinical trial by the pharmaceutical company is, for example, a contract research organization (CRO). The person in charge of the institution entrusted with the clinical trial by the pharmaceutical company is, for example, a clinical research associate (CRA).

The terminal device 20 outputs the selection criterion to, for example, an acquisition unit 11 of the clinical trial support device 10. The terminal device 20 acquires information about the patient to be clinically tested from an output unit 15 of the clinical trial support device 10. The terminal device 20 outputs information about the patient to be clinically tested to a display device (not illustrated).

The data management device 30 is a device that stores data regarding the treatment of a patient. The data regarding the treatment of the patient is, for example, data of an electronic medical record input by a doctor. The information in the electronic medical record may be data input by a nurse, a laboratory technician, a physical therapist, or a counselor. The data regarding the treatment of the patient may be information of one or more items of patient disease information, complication information, biomarkers, disease status, guideline scores, medical histories, efficacy, and test results other than data described in the electronic medical record. The data management device 30 outputs data regarding the treatment to the acquisition unit 11 of the clinical trial support device 10, for example.

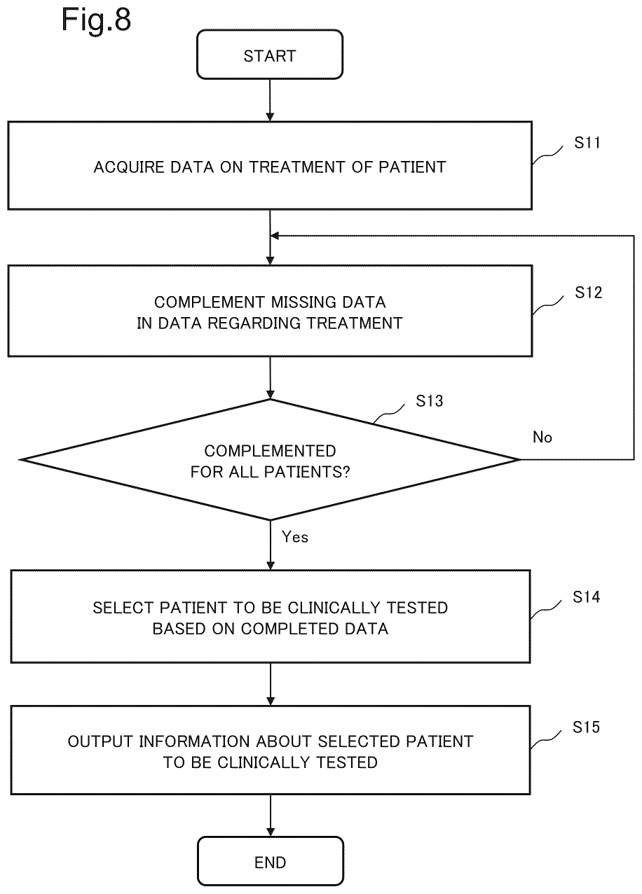

The operation of the clinical trial support device 10 in the process of extracting the patient to be clinically tested will be described. FIG. 8 illustrates an example of a flow in a process in which the clinical trial support device 10 extracts a patient to be clinically tested.

The acquisition unit 11 acquires data regarding the treatment of a patient (step S11).

When the data regarding the treatment of the patient is acquired, the complementing unit 12 complements missing data in the data regarding the treatment among the data to be used for selecting the patient to be clinically tested (step S12).

In a case where the data has been complemented for all the target patients (Yes in step S13), the selection unit 13 selects the patient to be clinically tested based on the complemented data (step S14).

When the patient to be clinically tested is selected, the output unit 15 outputs information about the selected patient to be clinically tested (step S15).

In a case where there is a patient whose data has not been complemented in step S13 (No in step S13), the process returns to step S12, and the complementing unit 12 complements missing data in the data regarding the treatment for the patient whose data has not been complemented.

The clinical trial support device 10 complements missing data in the data regarding the treatment among data used to select a patient to be clinically tested. The clinical trial support device 10 selects a patient to be clinically tested based on the complemented data. The clinical trial support device 10 outputs information about the selected patient to be clinically tested. As described above, by selecting the patient to be clinically tested based on the complemented data, for example, the number of patients that can be selected can be increased, so that the clinical trial support device 10 can efficiently select the patient to be clinically tested.

The clinical trial support device 10 can improve the effectiveness of clinical trial data in a clinical trial performed on the selected patient to be clinically tested, for example, by selecting the patient to be clinically tested in such a way that the selected patient to be clinically tested is not biased.

By selecting the patient in the control group based on data regarding the past treatment, it is possible to conduct a clinical trial by selecting only the patient in the clinical trial group. Therefore, the clinical trial support device 10 can improve the efficiency of selecting the patient to be clinically tested. By selecting the patient in the control group based on data regarding the past treatments, administration of placebo can be suppressed, and thus the clinical trial support device 10 can improve the quality of medical care.

By selecting a patient to be clinically tested based on the complemented data, the patient to be clinically tested can be selected from among patients with data missing, and thus the clinical trial support device 10 can select a patient to be clinically tested even in a medicine clinical trial for an injuries and sickness with few cases. By selecting a patient in the control group based on data regarding the past treatment, a patient in progress of treatment can be selected as a patient in the clinical trial group. Therefore, the clinical trial support device 10 can select a patient to be clinically tested even in a medicine clinical trial for an injuries and sickness with few cases.

For example, by selecting a patient to be clinically tested based on the complemented data, the patient to be clinically tested can be selected from among patients with data missing, and thus the clinical trial support device 10 can widely extract the patients to be clinically tested. Therefore, a person in charge of selecting a patient to be clinically tested can select a patient to be clinically tested from a wide range of patients selected based on the complemented data, for example, and can appropriately make a decision on selection of a patient to be clinically tested. Therefore, the clinical trial support device 10 can support decision-making regarding selection of a patient to be clinically tested.

Each processing in the clinical trial support device 10 may be executed in a distributed manner in a plurality of information processing devices connected via a network. For example, the processing in the complementing unit 12 and the selection unit 13 may be performed in another information processing device. Which information processing device performs each process in the clinical trial support device 10 can be appropriately set.

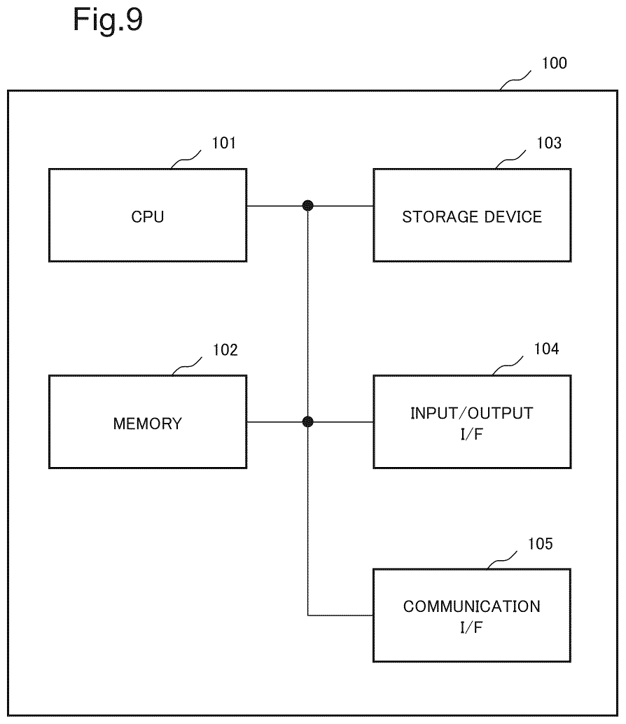

Each process in the clinical trial support device 10 can be implemented by executing a computer program on a computer. FIG. 9 illustrates an example of a configuration of a computer 100 that executes a computer program for executing each process in the clinical trial support device 10. The computer 100 includes a central processing unit (CPU) 101, a memory 102, a storage device 103, an input/output interface (I/F) 104, and a communication I/F 105.

The CPU 101 reads and executes a computer program for executing each processing from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores a computer program executed by the CPU 101 and data being processed. The storage device 103 stores a computer program executed by the CPU 101. The storage device 103 includes, for example, a nonvolatile semiconductor storage device. As the storage device 103, another storage device such as a hard disk drive may be used. The input/output I/F 104 is an interface that receives an input from an operator to output display data and the like. The communication I/F 105 is an interface that transmits and receives data to and from the terminal device 20, the data management device 30, and other information processing devices. The terminal device 20 and the data management device 30 can also be configured as in the computer 100.

The computer program used for executing each processing can be stored in a computer-readable recording medium that non-transiently records data and distributed. As the recording medium, for example, a magnetic tape for data recording or a magnetic disk such as a hard disk can be used. As the recording medium, an optical disk such as a compact disc read only memory (CD-ROM) can also be used. A non-volatile semiconductor storage device may be used as a recording medium.

In a medicine clinical trial, a person in charge of selecting a candidate for a patient to be clinically tested extracts a patient conforming to the criterion for selecting the clinical test patient, for example, by checking the description of the medical record. For example, the person in charge determines whether the selection criterion match the content described in the medical record. The person in charge extracts a patient in which selection criterion and the content described in the medical record match as a candidate for the patient to be clinically tested. The person in charge is required to check the medical records of many patients and search for a patient that meets the selection criterion. Therefore, a system that supports selection of a candidate for a patient to be clinically tested may be used for selecting a candidate for a patient to be clinically tested. However, the techniques described in the background may require a lot of work to select the patient to be clinically tested.

Therefore, in order to solve the above problems, an object of the present disclosure is to provide a clinical trial support device and the like capable of efficiently selecting a patient to be clinically tested.

By using the extraction system or the like of the present disclosure, for example, a patient to be clinically tested can be efficiently selected.

Some or all of the above example embodiments may be described as the following Supplementary Notes, but are not limited to the following.

### Supplementary Note 1

A clinical trial support device including
an acquisition unit that acquires data regarding a treatment of a patient,
a complementing unit that complements missing data in the data regarding the treatment among data used for selecting a patient to be clinically tested using a graph indicating a relationship between patients generated based on the data regarding the treatment of the patient,
a selection unit that selects a patient to be clinically tested based on the complemented data, and
an output unit that outputs information about the selected patient to be clinically tested.

### Supplementary Note 2

The clinical trial support device according to Supplementary Note 1, wherein
the complementing unit complements missing data in the data regarding the treatment using a graph indicating a relationship between patients generated based on the data regarding the treatment of the patient.

### Supplementary Note 3

The clinical trial support device according to Supplementary Note 1, wherein
the complementing unit complements the missing data using non-structural data among the data regarding the treatment.

### Supplementary Note 4

The clinical trial support device according to Supplementary Note 1, wherein
the complementing unit generates a feature vector of each patient as data obtained by complementing the missing data, based on a graph indicating a relationship between patients generated based on the data regarding the treatment of the each patient and a criterion for selecting a patient to be clinically tested.

### Supplementary Note 5

The clinical trial support device according to any one of Supplementary Notes 1 to 3, wherein
the complementing unit complements missing data in the data regarding the treatment among data used for calculating goodness of fit to a criterion for selecting a patient to be clinically tested.

### Supplementary Note 6

The clinical trial support device according to Supplementary Note 5, wherein
the selection unit selects a patient to be clinically tested from among patients in which the selection criterion is met at a past time point and data regarding a treatment for an implementation period of a clinical trial from a time point at which the selection criterion is met is recorded.

### Supplementary Note 7

The clinical trial support device according to any one of Supplementary Notes 1 to 3, wherein
the selection unit selects the patient to be clinically tested further based on data regarding a treatment of a selected patient as the patient to be clinically tested.

### Supplementary Note 8

The clinical trial support device according to any one of Supplementary Notes 1 to 3, further including
a prediction unit that predicts data regarding a treatment at a predetermined time point based on the data regarding the treatment.

### Supplementary Note 9

The clinical trial support device according to Supplementary Note 7, wherein
the selection unit selects at least some of patients in a control group based on data of selected patients in a clinical trial group as the patients to be clinically tested.

### Supplementary Note 10

The clinical trial support device according to Supplementary Note 7, wherein
the selection unit further selects a patient in a clinical trial group based on data of selected patient in a clinical trial group as the patients to be clinically tested.

### Supplementary Note 11

The clinical trial support device according to Supplementary Note 4, wherein
the selection unit selects a patient to be clinically tested based on the feature vector of each patient.

### Supplementary Note 12

The clinical trial support device according to Supplementary Note 8, wherein
the complementing unit complements missing data in the data regarding the treatment predicted by the prediction unit.

### Supplementary Note 13

The clinical trial support device according to Supplementary Note 8, wherein
the prediction unit predicts data regarding a treatment at the predetermined time point for the patient to be clinically tested selected by the selection unit.

### Supplementary Note 14

The clinical trial support device according to Supplementary Note 8, wherein
the predetermined time point is a start time point of a clinical trial or a time point during an implementation period of the clinical trial.

### Supplementary Note 15

A clinical trial support method including
acquiring data regarding a treatment of a patient,
complementing missing data in the data regarding the treatment among data to be used for selecting a patient to be clinically tested,
selecting a patient to be clinically tested based on the complemented data, and
outputting information about the selected patient to be clinically tested.

### Supplementary Note 16

A non-transitory recording medium that records a clinical trial support program for causing a computer to execute the steps of
acquiring data regarding a treatment of a patient,
complementing missing data in the data regarding the treatment among data to be used for selecting a patient to be clinically tested,
selecting a patient to be clinically tested based on the complemented data, and
outputting information about the selected patient to be clinically tested.

Some or all of the configurations described in Supplementary Notes 2 to 14 dependent on the above-described Supplementary Note 1 can also depend on Supplementary Note 15 and Supplementary Note 16 by the same dependency relationship as Supplementary Notes 2 to 14. Furthermore, some or all of the configurations described as the Supplementary Notes can be similarly dependent on not only the Supplementary Notes 1, 15, and 16, but also various pieces of hardware, software, and various recording means or systems for recording software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A clinical trial support device comprising:
at least one memory storing instructions; and
at least one processor configured to access the at least one memory and execute the instructions to:
acquire data regarding a treatment of a patient;
complement missing data in the data regarding the treatment among data used for selecting a patient to be clinically tested using a graph indicating a relationship between patients generated based on the data regarding the treatment of the patient;
select a patient to be clinically tested based on the complemented data; and
output information about the selected patient to be clinically tested, wherein
the graph indicating the relationship between the patients is a graph in which nodes indicating respective patients are connected by edges connecting similar patients,
the graph indicating the relationship between the patients is generated using a graph generation model, and
the graph generation model is generated by performing deep learning using a neural network based on a relationship between data regarding a treatment of each patient and a graph indicating a relationship between patients.

2. The clinical trial support device according to claim 1, wherein
the at least one processor is further configured to execute the instructions to:
complement the missing data using non-structural data among the data regarding the treatment.

3. The clinical trial support device according to claim 1, wherein
the at least one processor is further configured to execute the instructions to:
generate a feature vector of each patient as data obtained by complementing the missing data, based on a graph indicating a relationship between patients generated based on the data regarding the treatment of the each patient and a criterion for selecting a patient to be clinically tested.

4. The clinical trial support device according to claim 3, wherein
the at least one processor is further configured to execute the instructions to:
convert the graph indicating the relationship between the patients and a goodness of fit to a criterion for selecting a patient to be clinically tested in each patient into a feature vector of each patient.

5. The clinical trial support device according to claim 1, wherein
the at least one processor is further configured to execute the instructions to:
complement missing data in the data regarding the treatment among data used for calculating goodness of fit to a criterion for selecting a patient to be clinically tested.

6. The clinical trial support device according to claim 5, wherein
the at least one processor is further configured to execute the instructions to:
select a patient to be clinically tested from among patients in which the selection criterion is met at a past time point and data regarding a treatment for an implementation period of a clinical trial from a time point at which the selection criterion is met is recorded.

7. The clinical trial support device according to claim 1, wherein
the at least one processor is further configured to execute the instructions to:
select the patient to be clinically tested further based on data regarding a treatment of a selected patient as the patient to be clinically tested.

8. The clinical trial support device according to claim 1, wherein
the at least one processor is further configured to execute the instructions to:
predict data regarding a treatment at a predetermined time point based on the data regarding the treatment.

9. The clinical trial support device according to claim 7, wherein
the at least one processor is further configured to execute the instructions to:
select at least some of patients in a control group based on data of selected patients in a clinical trial group as the patients to be clinically tested.

10. The clinical trial support device according to claim 7, wherein
the at least one processor is further configured to execute the instructions to:
select a patient in a clinical trial group based on data of a selected patient in a clinical trial group as the patient to be clinically tested.

11. The clinical trial support device according to claim 3, wherein
the at least one processor is further configured to execute the instructions to:
select a patient to be clinically tested based on the feature vector of each patient.

12. The clinical trial support device according to claim 8, wherein
the at least one processor is further configured to execute the instructions to:
complement missing data in the predicted data regarding the treatment.

13. The clinical trial support device according to claim 8, wherein
the at least one processor is further configured to execute the instructions to:
predict data regarding a treatment at the predetermined time point for the selected patient to be clinically tested.

14. The clinical trial support device according to claim 8, wherein
the at least one processor is further configured to execute the instructions to:
the predetermined time point is a start time point of a clinical trial or a time point during an implementation period of the clinical trial.

15. A clinical trial support method comprising:
acquiring data regarding a treatment of a patient;
complementing missing data in the data regarding the treatment among data to be used for selecting a patient to be clinically tested;
selecting a patient to be clinically tested based on the complemented data; and
outputting information about the selected patient to be clinically tested, wherein
the graph indicating the relationship between the patients is a graph in which nodes indicating respective patients are connected by edges connecting similar patients,
the graph indicating the relationship between the patients is generated using a graph generation model, and
the graph generation model is generated by performing deep learning using a neural network based on a relationship between data regarding a treatment of each patient and a graph indicating a relationship between patients.

16. A non-transitory recording medium that records a clinical trial support program for causing a computer to execute the steps of:
acquiring data regarding a treatment of a patient;
complementing missing data in the data regarding the treatment among data to be used for selecting a patient to be clinically tested;
selecting a patient to be clinically tested based on the complemented data; and
outputting information about the selected patient to be clinically tested, wherein
the graph indicating the relationship between the patients is generated using a graph generation model, and
the graph generation model is generated by performing deep learning using a neural network based on a relationship between data regarding a treatment of each patient and a graph indicating a relationship between patients.
